(19) United States
(12) Patent Application Publication
(10) Pub. No.: US 2010/0268217 A1
(43) Pub. Date: Oct. 21, 2010

(54) VESSEL SEALING DEVICE AND METHODS

(75) Inventor: Nagy Habib, London (GB)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: EMCISION LIMITED, London (GB)

(21) Appl. No.: 12/301,895

(22) PCT Filed: May 23, 2007
(86) PCT No.: PCT/GB2007/001914

§ 371 Date: Jun. 16, 2010

(30) Foreign Application Priority Data
May 24, 2006 (GB) 0610489.7
Jan. 11, 2007 (GB) 0700553.1

(51) Int. Cl. A61B 18/18

(52) U.S. Cl. 606/33

(57) ABSTRACT

A device is provided that is suitable for percutaneous insertion into a hollow vessel, such as a blood vessel, within the body of a patient for purpose of causing endoluminal closure of the vessel at a specified therapeutic site in the body of a patient. The device suitably is in the form of a catheter that is slidably mounted on a guidewire. The catheter may comprise one or more heating modules, as well as one or more extendable structures located on the device and optionally on the associated guidewire, that lead thermal ablation of the vessel walls and subsequent collapse of the vessel. The catheter can function alone or in cooperation with an associated guidewire to induce sealing of the vessel. Methods of using the catheter to treat lesions such as tumours or hemorrhages are also described.

### FIELD

The invention relates to apparatus and methods for performing percutaneous catheter-based interventional surgery. In particular the invention relates to methods and apparatus for causing endoluminal closure of hollow anatomical structures such as blood vessels.

### BACKGROUND

In many medical conditions such as arterio-venous vascular malformations and varicose veins, it is advantageous to block a blood vessel. In treating liver disease it is possible to induce liver regeneration by directing blood supply from one area to another, for example by blockage of portal blood to the right liver to induce hypertrophy of the left liver. Blocking blood flow can be used in the field of oncology, specifically in the field of treating tumours. One method of treating tumours is to interrupt the blood supply to the tumour. In many tumours there are a small number of discrete vessels supplying blood to the tumour. Blocking these vessels will cease the supply of nutrients to the tumour causing the tumour cells to die. Blood vessels that supply tumours can also be used to introduce an ablation catheter into the tumour.

Percutaneous surgical procedures involve insertion of a therapeutic probe, typically a catheter mounted on a guidewire, through an incision made in the skin of the patient. The probe can be guided to a therapeutic site in the body via the circulatory system of arteries and veins, thereby reducing the need to cause more extensive trauma to the patient by adopting more traditional open surgical techniques.

Prior methods for occluding blood vessels include injecting a sealing compound into the vessel, or positioning a plug or obstructive stent into the vessel. These have the disadvantage that these blocking structures may become displaced over time, and permit blood flow through the vessel. In some cases the structure may move to another vessel and cause an embolism.

Sealing of veins, particularly varicose veins, is described in US Patent Publication No. 2002/0143325 (Sampson et al.). A catheter is described that can be inserted into the vein and which comprises an array of radiofrequency (RF) electrodes flanked by expandable balloon structures located proximally and distally to the electrode array. In use, the catheter is positioned in the vein that is to be sealed, the proximal and distal balloons are expanded to induce occlusion of the vein and then blood is aspirated from the partitioned region between the balloons via perforations interspersed between the electrodes in the RF array. Once the blood has been removed from the partitioned region, the RF power is applied using the electrode array and closure of the vein is caused due to thermal ablation of the tissue in the vessel walls. The Sampson et al. device while suitable for sealing larger vessels such as the saphenous vein, is however unsuitable for use with smaller vessels, particularly vessels leading to tumours, due to the relatively large diameter of the device which is needed to accommodate the balloon distension, RF power, guidewire and blood aspiration conduits.

A catheter probe arrangement with bipolar RF electrodes has been described in International Patent Publication No. WO-96/36282 (Pecor et al.; Baxter International Inc.) for use in sealing the entry port or puncture wound left after percutaneous surgical procedures. However, the apparatus described in Pecor is directed at cauterisation of the relatively large entry wound which is close, or proximal, to the operator of the device. Pecor does not consider therapeutic applications that are remote from the location of the puncture wound. The preferred operative position of the Pecor device is outside of the blood vessel, within the adjacent tissue, and Pecor is concerned solely with the final closure stages of a procedure instead of at the therapeutic stage of surgical procedure itself.

In common with the above, RF ablation catheters in general have been restricted in use to ligation/closure of larger hollow anatomical structures. Not least because to ensure complete sealing and closure of the vessels typically requires that the surrounding tissue is physically compressed at the time of ablation so as to ensure good contact between the electrode surface and the vessel walls. When considering more delicate surgical procedures such as closure of blood vessels supplying a lesion (e.g. a tumour or hemorrhage) in abdominal organs, thorascic tissue or in the brain, it is clear that physical compression may not be possible or suitable. As a result, many thermal ablation catheters have not been routinely used for surgical intervention outside of the field of varicose vein treatment.

Hence, there exists a need for a device which can be used induce endoluminal closure of hollow anatomical structures such as blood vessels of a range of diameters from large to small. In addition, there exists a need for such devices that can be used percutaneously and targeted to sites within the body of a patient that are remote from the operator and which can reliably cause endoluminal closure, or sealing, of blood vessels at those sites.

### SUMMARY

In a first aspect the invention provides a device suitable for percutaneous insertion into a hollow vessel for purpose of causing endoluminal closure of the vessel at a specified therapeutic site in the body of a patient, comprising:
an elongate body having a distal end and a proximal end, the distal end comprising a distal tip portion, and a central lumen extending along at least a portion of the length of the elongate body, wherein the central lumen is configured to enable slidable mounting of the device upon a prelocated guidewire;
the distal tip portion comprising at least one heating module capable of heating the walls of the hollow vessel to a temperature that causes endoluminal closure of the hollow vessel; and
wherein the distal tip portion further comprises at least one extendable element that can be deployed outwardly the elongate body so as to contact and/or penetrate the walls of the hollow vessel.

In a second aspect the invention provides a device suitable for percutaneous insertion into a hollow vessel for purpose of causing endoluminal closure of said vessel at a specified therapeutic site in the body of a patient comprising:
a guidewire having a distal end and a proximal end, the distal end comprising a first distal tip portion, and wherein the distal tip portion comprises a first RF electrode located at a position adjacent and immediately proximal to the distal end; and
an elongate body having a distal end and a proximal end, the distal end comprising a second distal tip portion, and a central lumen extending along a least a portion of the length of the elongate body, wherein the central lumen is configured to enable slidable mounting of the elongate body upon the guidewire, and the second distal tip portion comprising a second RF electrode;
   wherein in use the guidewire and elongate body are juxtaposed such that upon application of RF energy the first and second RF electrodes are capable of cooperating to cause heating the walls of the hollow vessel to a temperature that causes endoluminal closure of the hollow vessel.

In a third aspect the invention provides a device suitable for percutaneous insertion into a hollow vessel for purpose of causing endoluminal closure of the vessel at a specified therapeutic site in the body of a patient comprising:
an elongate body having a distal end and a proximal end, the distal end comprising a distal tip portion, and a central lumen extending along a least a portion of the length of the elongate body; and
the distal tip portion comprising a monopolar RF electrode, which in cooperation with a remotely located electrode, is capable of heating the walls of the hollow vessel to a temperature that causes endoluminal closure of the hollow vessel;
wherein the RF electrode is between about 2 mm and about 20 mm in length.

In a fourth aspect the invention provides a method for endoluminal closure a blood vessel at a predetermined site within the body of a patient, the predetermined site being within or adjacent to the site of a lesion in tissue that is supplied by the blood vessel, the method comprising:
(a) introducing into the blood vessel a guidewire at a site remote from the predetermined site within the body of the patient, the guidewire having a distal tip, and directing the distal tip of the guidewire to a location substantially within the vicinity of the predetermined site;
(b) introducing onto the guidewire via a slidable mounting, a catheter, wherein the catheter comprises a distal tip region comprising at least one heating module located thereon;
(c) directing the distal tip region of the catheter to the predetermined site within the body of the patient by tracking the catheter along the guidewire;
(d) applying energy to the walls of the blood vessel via the heating module such that the tissue is heated to a point that causes endoluminal closure of the blood vessel;
(e) monitoring the energy application in step (d);
(d) ceasing application of energy when endoluminal closure has been completed; and
(e) withdrawing the catheter and guidewire from the closed blood vessel.

### DRAWINGS

The invention is further illustrated by reference to the accompanying drawings in which:

FIG. 1 shows the prior art solution in which a blockage is inserted into an blood vessel that leads to a tumour mass.

FIG. 2(a) shows a diagrammatic side view of an embodiment of the invention in which the catheter is inserted into a vessel and comprises two cylindrical members that act as bipolar electrodes that enable the application of RF energy to the surrounding tissue. (b) shows an oblique cutaway view of a similar embodiment to that shown in (a) in which the internal components of the distal tip portion of the catheter including the presence of a temperature sensor are visible.

FIG. 3 (a) shows a cross sectional diagrammatic side view of an embodiment of the invention in which the catheter includes a vessel occlusion structure, in the form of a fluid inflatable bladder located proximal to the bipolar electrodes, thereby allowing for blockage of the vessel that is to be sealed. (b) shows an oblique cutaway view of a similar embodiment to that shown in (a), with the expandable bladder assuming a frustoconical conformation upon deployment.

FIG. 4 shows a diagrammatic side view of an alternate embodiment of the invention, where the bipolar electrodes include extendable elements in the form of flexible arms that extend outwardly from the catheter and are able to make direct contact with the surrounding vessel wall following proximal retraction of an outer sheath.

FIG. 5 shows a diagrammatic side view of an alternate embodiment of this invention where the energy is delivered using a microwave dipole antenna.

FIG. 6 shows a diagrammatic side view of an alternate embodiment of this invention where the energy is delivered using a cylindrical ultrasound array, the cutaway section demonstrates the laminar construction of the cylindrical array.

FIG. 7 shows a diagrammatic side view of an alternate embodiment of this invention where the energy is delivered using a rotating focused ultrasound transducer.

FIG. 8 shows a diagrammatic side view of an alternate embodiment of this invention where the energy is delivered using a laser beam.

FIG. 9 shows an oblique cutaway view of an embodiment of the invention in which a monopolar RF configuration is adopted for the catheter, with the electrode located at the distal tip of the catheter. The catheter is also slidably loaded onto a guidewire. In this embodiment the guidewire further comprises an expandable structure proximally adjacent to the distal tip that can function as an RF electrode.

FIG. 10 (a) shows an oblique view of the distal tip of the embodiment of the guidewire of the invention shown in FIG. 9 which comprises an expandable electrode in the undeployed state. (b) shows the electrode in the deployed or expanded state forming a basket-like structure. (c) Shows an axial view along line Y in FIG. 10 (b). (d) shows an axial view along line Y in FIG. 10 (a). (e) shows a side view of the guidewire with the electrode in the expanded state, in an additional embodiment a helical shaft is provided that surrounds the exterior surface of the guidewire. (f) shows a side view of the guidewire with the electrode in the unexpanded state.

FIG. 11 shows an oblique view of the distal tip of the alternate embodiment of the guidewire of the invention in which the guidewire further comprises an expandable structure that when deployed takes the form of a double helix coil electrode.

FIG. 12 shows a cut away side view of the proximal end of an embodiment of the catheter of the invention which provides the electrical connection to the guidewire and a user interface in the form of a hub, (a) shows the configuration of the hub in which the plug and socket means are engaged to allow electrical connection. (b) shows the configuration of the hub in which the plug and socket means are separated so that no electrical connection is made.

FIG. 13 shows a side view of the distal tip of an alternate embodiment of the guidewire of the invention in which the guidewire includes an expandable structure located proximally to the distal tip that is shown in the process of deploying in (a) to (c). (d) shows the expanded structure within a vessel, demonstrating the increased contact between the flexible electrode arms and the vessel wall.

FIG. 14 shows a side view of the distal tip of the embodiment of the guidewire of the invention as shown in FIG. 14, (a) displays the further inclusion of additional radial electrode wires that retractably extend across the span between the expanded flexible electrode arms. (b) shows an additional embodiment of the invention in which a resilient member is located at the proximal end of the expandable structure.

FIG. 15 shows a diagrammatic side view of an embodiment of the invention where a monopolar electrode includes flexible arms that can protrude outwardly from the catheter via apertures and are able to make direct contact with and penetrate into the tissue in the surrounding vessel wall following proximal retraction of an outer sheath.

FIG. 16 shows a diagrammatic side view of an embodiment of the invention where a bipolar electrode includes flexible arms that can protrude outwardly from the catheter and are able to make direct contact with and penetrate into the tissue in the surrounding vessel wall following proximal retraction of an outer sheath.

FIG. 17 shows a cross sectional side view of an embodiment of the invention providing further detail relating to deployment of the tissue penetrating arms such as those shown in FIGS. 15 and 16.

FIG. 18 shows a cross sectional side view of an alternative embodiment of the invention providing further detail relating to deployment of the tissue penetrating arms such as those shown in FIGS. 15 and 16.

FIG. 19 shows a diagrammatic side view of an embodiment of the invention in which (a) the catheter is loaded onto a guidewire and tracked to the site requiring therapy within the patient's body. (b) the catheter comprises a side port with a hinged gate that can open inwardly, along arrow P, into the central lumen of the catheter when the guidewire is withdrawn proximally, and act as a deflector to direct the reinserted guidewire outwardly so that it will penetrate the wall of the surrounding vessel.

FIG. 20 shows photographs of samples of bovine liver tissue showing the heating pattern (ablation) caused following application of RF energy to the tissue utilising (a) a bipolar catheter configuration of the invention, the scale bar at the top right indicates a distance of 10 mm. The heating pattern indicated by arrow E is due to a bipolar separation of 7 mm between the RF electrodes. The heating pattern shown by arrow F is due to a bipolar separation of 10 mm between the RF electrodes. (b) a monopolar configuration is adopted with a separately located grounding pad (not shown). Arrow G shows the heating pattern obtained and the scale bar at the bottom indicates a distance of 10 mm.

### DETAILED DESCRIPTION

Unless stated otherwise the terms used herein have the same meanings as those understood by a person of appropriate skill in the art. All cited documents are herein incorporated by reference in their entirety.

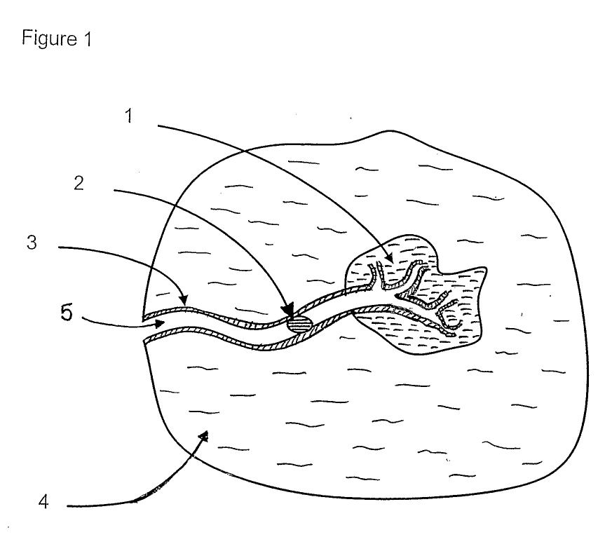

The prior art solution is shown in FIG. 1. An organ 4, may contain a region of tissue comprising a lesion 1 that requires therapy. The lesion may be a solid tumour (malignant or benign), a hemorrhage, a diseased tissue, hypertrophic tissue, a varicosity or other tissue where it is desired that blood supply be reduced or interrupted. The tissue region 1 is supplied by a blood vessel 5 having walls 3, typically an artery or an arteriole. An obstruction 2 can be inserted into the vessel to interrupt the blood supply to the lesion 1. A problem encountered with this approach is that the obstruction be displaced as a result of blood pressure or movement of the patient to block a different vessel, or permit flow through the vessel 3 to the tumour.

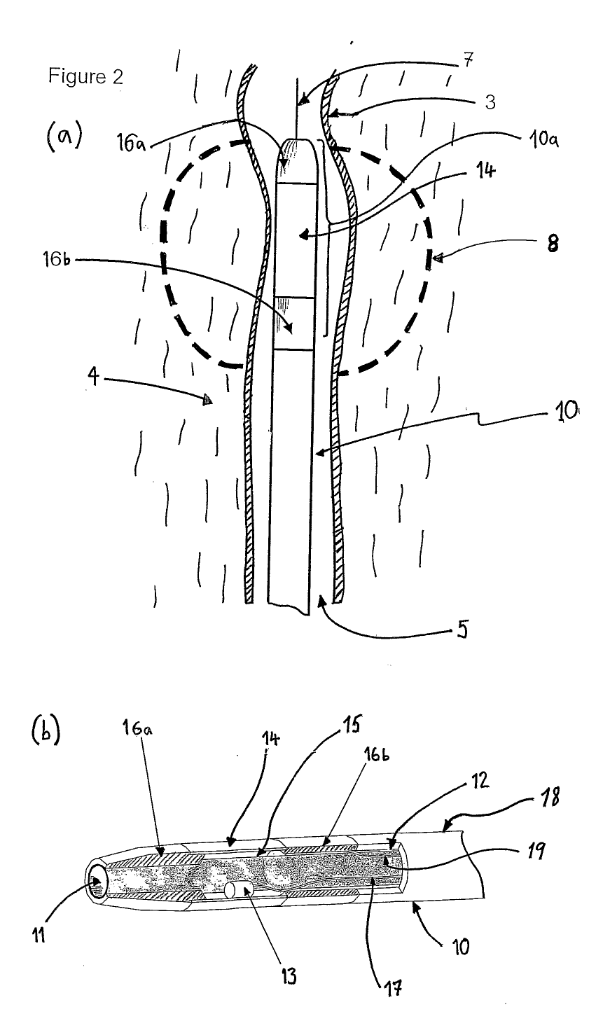

A first embodiment of the invention is a device comprising the portion shown in FIG. 2. According to the invention a flexible elongate catheter 10 includes a proximal end where control of the device by the user is administered, an elongate flexible rod-like portion and a tip portion at its distal end. The distal end of the catheter is typically located at the site within the body of the patient where therapy is to be administered. The catheter comprises a tip portion 10a that can comprise a radio-opaque material to enhance the ability to visualise the tip in vivo and direct therapy to the correct location. As shown in FIG. 2(b), the tip portion of the catheter 10a comprises two cylindrical electrodes, a distal electrode 16a and a proximal electrode 16b. The electrodes are connected to opposite polarities of an RF generator. RF current will flow between the electrodes 16a and 16b, and will intercept the wall of the vessel 3. This current causes heating and, thus, depending upon the distance between the electrodes can result in ablation of a spherical zone of tissue 8 between the electrodes and will heat the vessel wall and the tissue surrounding the vessel. The electrodes may be in contact with the vessel wall.

Typically the catheters of the invention are operated according to three main phases of therapy: an insertion phase, a therapy phase and a removal phase. The insertion phase includes the percutaneous insertion of the guidewire (if required) and the location of the guidewire and/or catheter to the site where therapy is to be administered. The therapy phase includes the steps of deploying the electrode (if necessary) and administering thermal ablation to the vessel, and optionally the surrounding tissue. The removal phase includes the withdrawal of the catheter and/or guidewire from the site of ablation, usually back along the initial insertion route. Optionally, the therapy phase and withdrawal phase can overlap such that ablation is applied along a portion of the vessel rather than simply at a single site.

The catheter 10 is optionally be deployed over a flexible guidewire 7. The RF current is suitably at a frequency between 100 kHz and 5 MHz. The catheter 10 can be used in two different modes. The catheter can be inserted into one or more vessels 5 that provide a blood supply a lesion 1, so the distal end 10a is positioned at any point in the vessel close to but upstream to the lesion 1. RF energy is then applied causing heating of the surrounding tissue, including collagen and other extracellular matrix components in the vessel wall 3, which causes the vessel 5 to collapse and prevent blood flow into the lesion 1.

In another mode the catheter 10 can be inserted into a vessel 5 in the centre of the lesion 1 and the RF energy is applied to also heat the surrounding tissue beyond the vessel wall 3. This embodiment of the invention is particularly suitable where the surrounding tissue in the lesion 1 is a tumour.

The catheter 10 may be connected to the RF energy in a number of different ways. In one embodiment the bipolar cylindrical electrode arrangement 16a,b may be connected to opposite polarities of an RF generator so RF current will flow between the proximal and distal electrodes 16a and b.

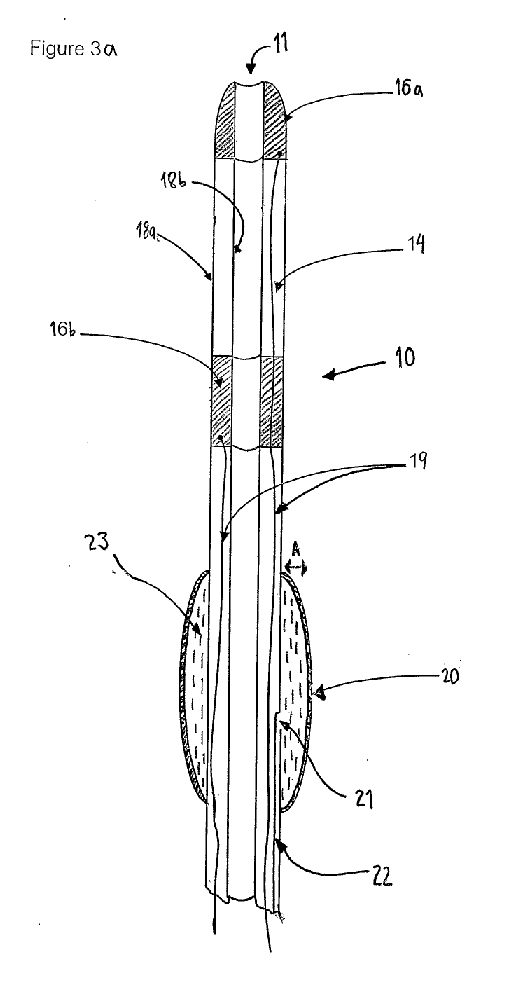

As shown in FIGS. 2b and 3a, the catheter 10 comprises an elongate body that is constructed with an outer wall 18a, and an inner wall 18b. The lumen 11 defined by the inner wall 18b will accept a guidewire so the catheter 10 may be loaded over a pre-located guidewire and directed to the site in the patient's body requiring therapy. The lumen 11 may extend substantially along the entire length of the catheter 10 (thereby facilitating an over-the-wire mounting on the guidewire) or along only a portion of the catheter 10 (thereby facilitating a monorail mounting on the guidewire). The catheter body is suitably manufactured from plastics or polymeric biocompatible materials known in the technical field.

The annular chamber 15 between the inner and outer walls houses wires 19 that allow connection of the external RF source to the electrodes 16a and 16b. The electrodes 16a/b are typically annular or collar-shaped members suitably constructed from a biocompatible metal selected from stainless steel; platinum; silver; titanium; gold; a suitable alloy and/or a shape memory alloy. The distance between the electrodes on the distal end region 10a will, to an extent, define the shape of the thermal ablation pattern and the extent of the penetration of energy into the surrounding tissue. Greater separation between the electrodes tends to result in two distinct foci or regions of thermal ablation, whereas closer spacing allows the areas of ablation to converge into a single elongated region. According to the invention, the distal and proximal electrodes are typically spaced no more than approximately 15 mm apart, and suitably anywhere between around 7 mm and about 10 or 12 mm apart.

The catheter tip 10a may be fixed in position within the vessel wall with an expandable occluding structure, such as an inflatable bladder or balloon 20. In this arrangement the balloon 20 can be inflated and deflated (along axis A shown in FIG. 3a) by injecting fluid 23 using techniques known from percutaneous angioplasty. The occluding structure serves to centre the catheter tip 10a within the vessel 5 and also temporarily obstructs the blood flow, to reduce cooling of the vessel wall 3 during the ablation phase of therapy. The conduit 22 comprises a channel that carries the fluid used to inflate and deflate the balloon 20 through the aperture 21 from an external source. The fluid 23 may be a liquid or a gas. In one embodiment of the invention the expanded balloon 20 is in the adopts a frustoconical configuration as shown in FIG. 3b.

In accordance with the invention, the catheter may include one or more extendable elements that can be expanded outwardly from the body of the catheter and contact the walls of the surrounding vessel. The extendable elements suitably cooperate with or be comprised within the heating module such that they serve to dissipate or conduct energy into the vessel walls and optionally the surrounding tissue, thereby enhancing the thermal ablation properties of the device. Suitable extendable elements can be selected from: a wire; an arm; a panel; and a needle. Outward expansion can be substantially radially relative to the longitudinal axis of the elongate body of the catheter, or can be substantially coaxially relative to the said longitudinal axis. Alternatively the outward expansion can be at an intermediate angle that is between the longitudinal axis and the radial axis that is perpendicular to the longitudinal axis, such as in a distal direction extending forwardly from the distal tip portion but outwardly into the surrounding tissue.

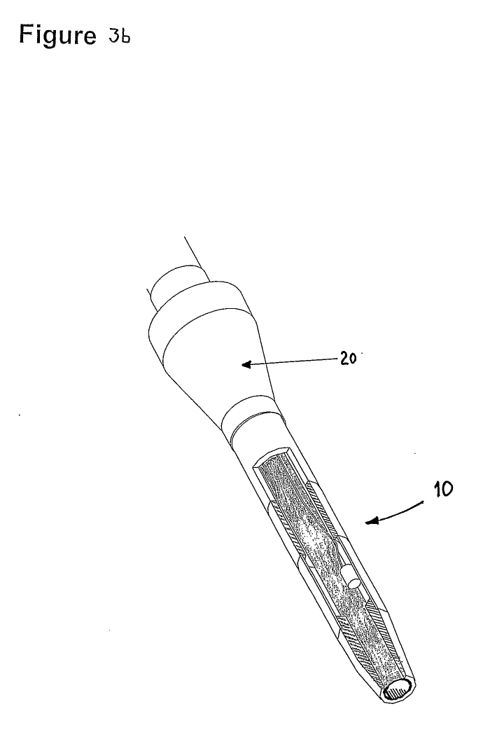

In a second embodiment of the invention, a catheter 10 (shown in FIG. 4) includes extendable elements in the form of flexible electrode tines or arms 31a and b that are retractably mounted on the distal and proximal electrodes 16 a and b. When in the undeployed state the arms 31 a and b can be withdrawn within the chamber 15 (not shown). Alternatively, an outer sleeve 30 mounted over the catheter tip can constrain the arms 31a and b, which can be made from a prestressed material or shape memory alloy, and hold them in a configuration that is substantially parallel with the longitudinal axis of the body of the catheter 10. When the sleeve 30 is retracted the arms 31a and b revert to their unstressed configuration—i.e. they extend substantially radially outwardly from their respective electrodes to make contact the vessel wall 3. In this way the arms 31a and b are able to conduct RF current directly to the vessel wall 3 and surrounding tissue. In use, the catheter 10 can be withdrawn from the vessel in a proximal direction (shown by arrow B) into the sleeve 30 thereby combining the application of an extended region of tissue ablation along the length of the vessel 3 together with the step of retracting the arms 31a and b after the therapy phase is over, which may assist in subsequent removal of the device from the patient's body.

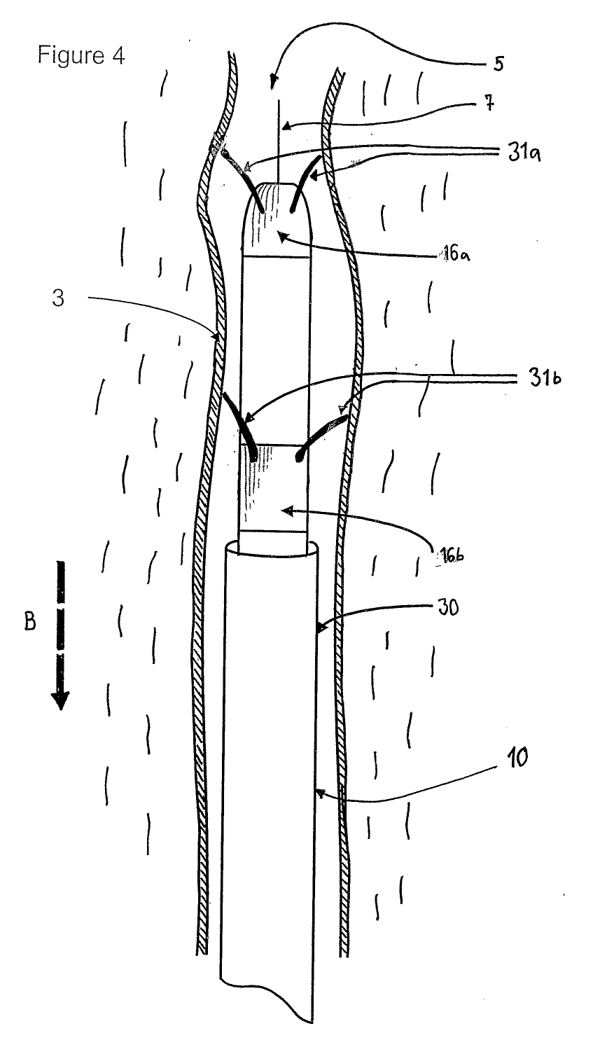

An alternate embodiment of the invention is shown in FIG. 5 in which the vessel wall 3 and optionally also the surrounding tissue is ablated using microwave energy. Two conducting cylinders of equal length 25a and b are mounted with a small interval between them such that they form a dipole antenna. The cylinders are connected to the inner conductor 26 and outer conductor 27 of a coaxial cable 28. The cable is supplied with microwave energy at frequencies between 200 MHz and 5 GHz. The length of the two cylinders is arranged to be approximately one half of the wavelength of the microwave radiation in tissue. When microwave energy is applied to the coaxial cable the dipole will act as a source of microwave radiation, which will propagate as a cylindrical wave, depositing heat in the region next to the catheter.

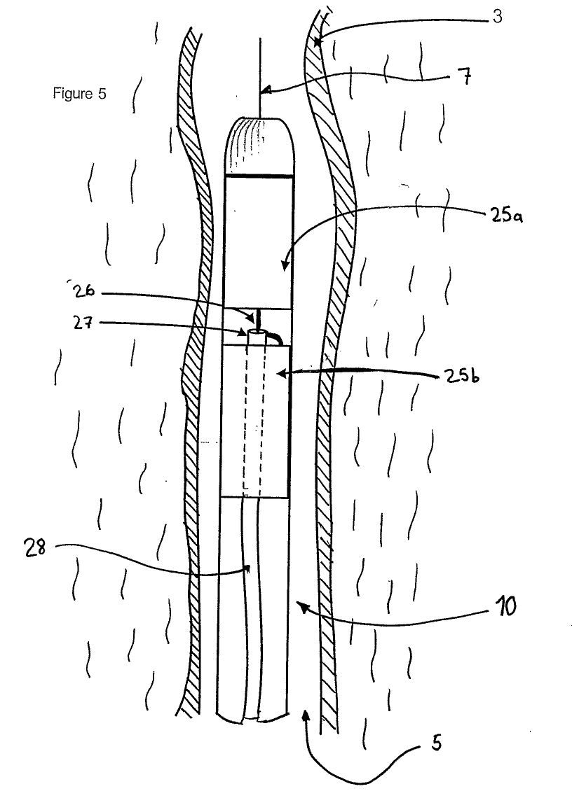

An alternate embodiment is shown in FIG. 6 in which the vessel wall is ablated using ultrasound energy. A cylinder 32 of a piezo-electric material such as PZT-4 is mounted on the catheter. Electrodes are plated on the inner 32a and outer 32b cylindrical surface of the cylinder 32. The electrodes may suitably be silver, gold, or a titanium or tungsten alloy. RF energy is applied between the electrodes by connecting to an external RF source via the connecting wires 33. This RF energy is at an ultrasound frequency, for example the energy will typically be between 200 kHz and 20 MHz. This generates cylindrical ultrasound wave which will radiate outwards. When the ultrasound propagates through an attenuating material such as the vessel wall 3, heat will be deposited in the vessel wall 3 causing sealing of the vessel.

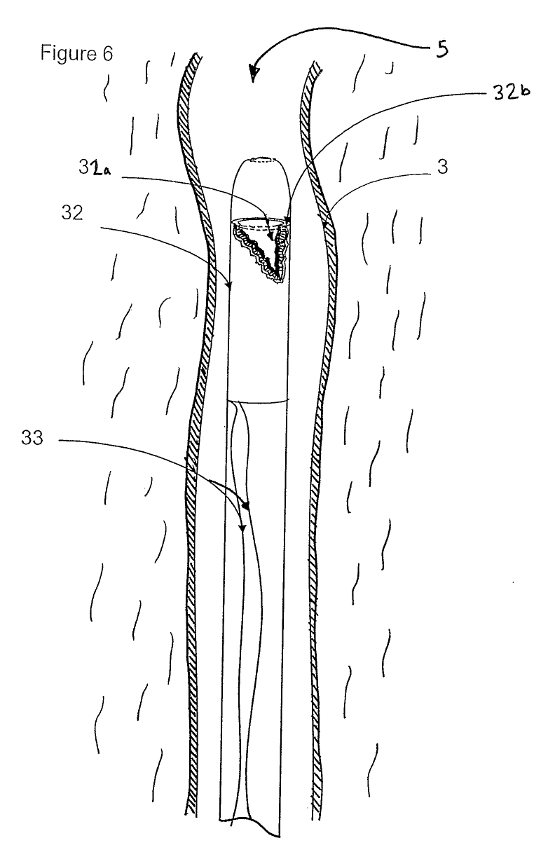

FIG. 7 shows an alternative embodiment of the invention, in which the vessel wall is heated using a focused ultrasound transducer 35, which generates a beam of ultrasound 36, which will deposit energy when it intercepts an attenuating material such as the vessel wall 3. The transducer is mounted on a plate 39 which is rotated using a drive shaft 37, to sweep the beam through 360° to heat the whole circumference of the vessel. The ultrasound transducer is housed in a fluid-filled cavity 38. The ultrasound material may suitably be constructed of a material such as PZT-4, and can be shaped into a concave bowl to focus the ultrasound energy.

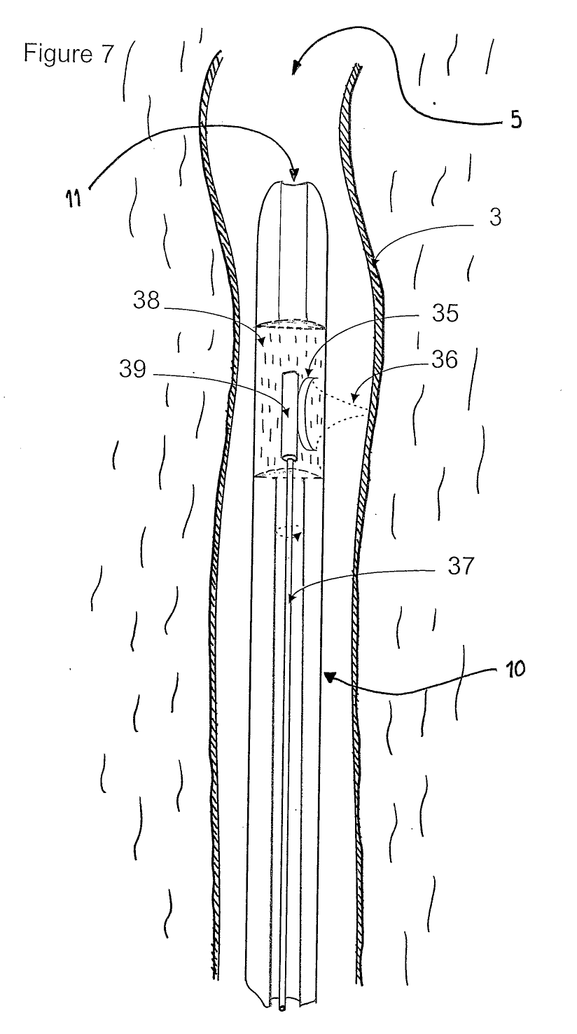

A specific embodiment is shown in FIG. 8 in which the vessel wall 3 is heated using a laser beam 41, which is transmitted along the inside of the catheter though an optical fibre 40. A mirror 42 directs the laser beam to be perpendicular the catheter so it deposits energy when it intercepts an opaque material such as the vessel wall 3. The mirror 42 may be made of the same material as the fibre, or another suitable transparent material such as glass, polymethylacrylate. The mirror 42 may be silvered, or rely on total internal reflection, between the interface of the transparent material and air. The mirror 42 and fibre 40 are rotated to sweep the laser beam through 360° to heat the whole circumference of the vessel. Alternatively the mirror 42 is a conical shape, to direct the laser beam to assume a disc configuration, this will not require the mirror 42 to be rotated.

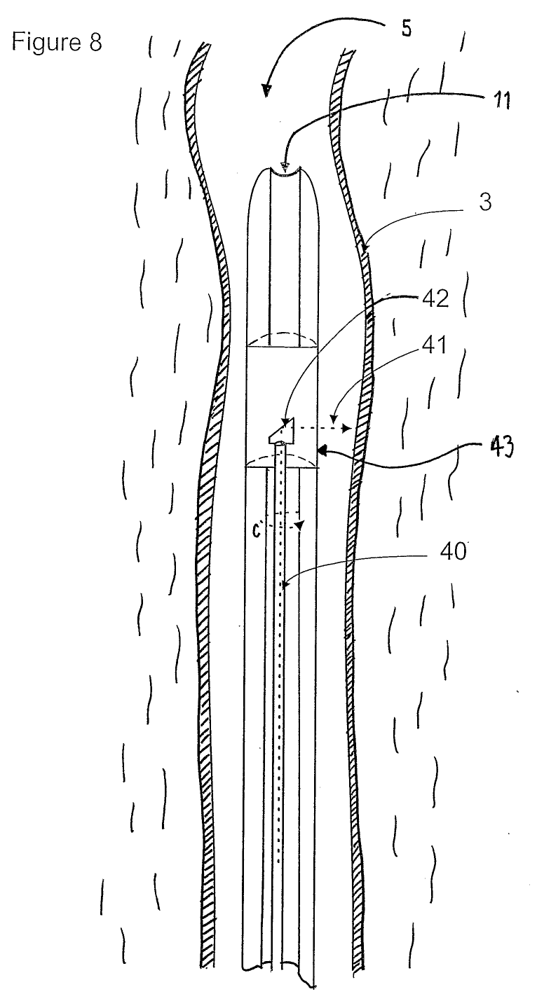

The embodiments of the invention described so far include description of a bipolar RF arrangement located on the catheter tip. In alternative embodiments of the invention described below, the catheter tip may include only a single RF electrode (a monopolar configuration) with the other electrode polarity provided by a grounding pad in contact with the patient's body. In yet another alternative embodiment of the invention, shown in FIG. 9, a distal electrode 56 is present on the catheter tip 50a may be connected to one polarity of an RF source. The guidewire 60 is either connected to the opposite polarity or an electrode 61 is provided on the guidewire 60 at a position close to the distal tip 62 of the guidewire 60. In use, the RF current flows from the distal electrode 56 on the catheter 50 to the guidewire 60 or guidewire electrode 61.

In accordance with an embodiment of the invention, the catheter may also contain an extended monopolar RF electrode arrangement in the distal tip portion of the catheter. The extended monopolar electrode may be as much as 20 mm in length, although typical size can vary depending upon the therapeutic need from about 2 mm to about 15 mm, and optionally around 10 mm. In one embodiment of the invention, distal and proximal RF electrode contacts are provided on the distal catheter tip in a arrangement similar to that seen with the bipolar electrode configuration, but wherein a thin layer of conductive material, such as a metal film or foil layer, extends between the distal and proximal electrode contacts. The conductive layer can also be synthesised via vapour deposition of a layer of conductive material, such as a metal, onto the surface of the catheter distal tip region or by encapsulating the region with a free standing foil layer. Alternative embodiments also include a flexible electrode configuration that comprise a helix, interconnected rings, or a stent-type structure. Materials suitable for use in manufacture of the conductive layer are substantially identical to those described herein for the manufacture of the RF electrodes. This extended monopolar configuration can be used in conjunction with an external grounding pad or with a guidewire mounted electrode to complete the RF circuit. Advantageously, the extended monopolar configuration allows for the distal tip portion to retain flexibility which is of great importance when positioning the device of the invention in a blood vessel as close as possible to the site of a lesion.

Observing the level of electrical impedance in the surrounding tissue is one way of monitoring the progress of the therapy/heating phase. For instance, electrical impedance can be monitored during heating and when a predefined threshold is reached the heating phase is deemed to have been completed. In an example of the invention in use, described in more detail below, the impedance threshold was set at increase in 10% above the starting level. It will be appreciated that the threshold will vary depending upon the type of tissue surrounding the catheter tip, as well the nature of the procedure (i.e. if thermal ablation of the surrounding tissue is required in addition to sealing of the vessel).

Improved monitoring is further provided by inclusion of temperature sensing means in the catheter tip 8. FIG. 9 also shows the catheter tip 50a of the invention further comprising a thermocouple temperature sensor 53. In the embodiments of the invention where the catheter tip comprises a bipolar RF electrode arrangement the temperature sensing means is conveniently located between the electrodes (see 13 in FIG. 2(b)). However, it will be appreciated that in alternative embodiments of the invention as described herein, it is simply preferred that the temperature sensing means be located on the catheter tip at a position close to where the ablation is to occur. Clearly it is desirable that the therapy administered is sufficient to induce closure of the vessel and optionally thermal ablation of the surrounding tissue. However it is not desirable to cause widespread and uncontrolled heating of potentially healthy tissue that is adjacent to the therapy site, hence, the option for improved control of the heating step.

As mentioned previously, it is advantageous to provide a temporary occluding structure on the catheter tip 50a, particularly to reduce the effect of blood flow that has the potential to cause cooling of the therapy site during the heating phase. FIG. 9 also shows an embodiment of the invention where the expanded occluding structure is in the form of a frustoconical expandable balloon 57. This configuration of balloon 57 provides advantages such as the reduced tendency for back flow during the therapy and withdrawal phases and improved contact characteristics between the occluding structure and the vessel wall. The temporary occluding structure may be deflated in a controlled or staged manner during the withdrawal phase in order to prevent a sudden increase in blood pressure at the ablation site that could cause failure of the seal or at worst rupture or hemorrhage of the therapy site.

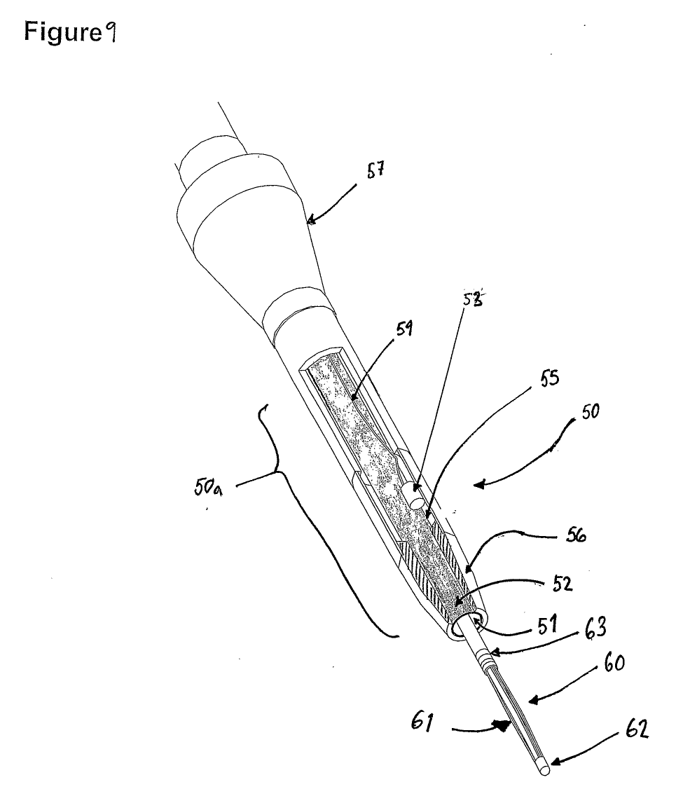

The use of a guidewire electrode, represents one particular embodiment of the present invention. In FIG. 10 one configuration of the guidewire electrode is shown which comprises an expandable structure both allowing for improved contact between the surface of the electrode and walls of the surrounding vessel as well as anchoring the guidewire in its location such that the catheter may be accurately guided to the therapy site. FIG. 10(a) shows a guidewire 60 comprising an electrode 61 that assumes an expandable basket structure, also referred to as a spring electrode, that includes deformable splines 61a secured at either end to a static tip 62 and a slidable collar 64. The guidewire further comprises an insulating sleeve 63 that extends along the length of the guidewire. When deployment of the electrode is required the slidable collar 64 is able to be moved slidably in the direction shown by arrow Z in FIG. 10(a) in order to reduce the longitudinal distance between the collar 64 and the tip 62 causing the deformable splines 61a to bow radially outward, as shown in FIG. 10(b). Views along the longitudinal axis of the guidewire are shown in FIGS. 10 (c-d) further showing the radial expansion of the spring electrode. In a specific embodiment of the invention an additional spring sheath in the form of a spiraled shaft is applied to at least a portion of the exterior of the insulating sleeve 63 at the location of the collar 64, which accommodates the need for expansion of the insulation when the collar 64 is slid towards the tip 62 (see FIGS. 10(e-f)). The expanded spring electrode is retracted following administration of therapy by initiating sliding of the collar 64 in the reverse direction, i.e. proximally, to that to arrow Z (FIG. 10(a)).

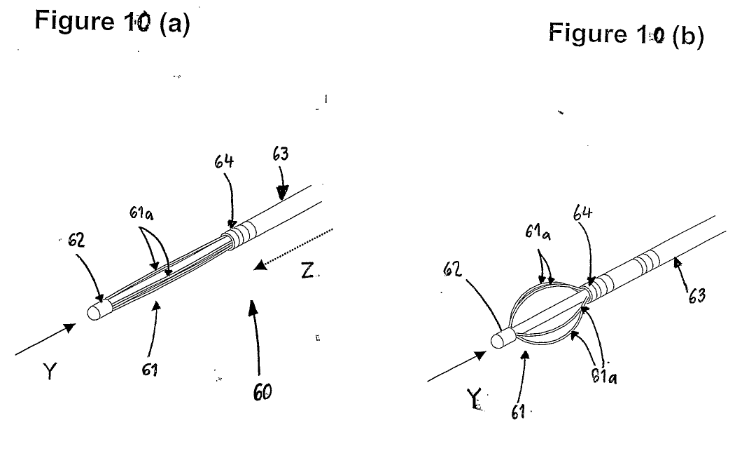

The expandable electrode need not be limited to the configuration described above and shown in FIG. 10. In FIG. 11 an alternative arrangement is shown in which the guidewire 70 comprises an expandable electrode 71 in which the electrode elements form of a helical or coil spring 71a. Operation of the expansion/retraction of the electrode 71 is substantially similar to that described previously. However, one advantage of the helical coil spring configuration is that it is possible to provide a bipolar RF electrode configuration solely on the guidewire by adopting a double helical structure wherein each element 71a of the helix provides the opposite polarity. In this embodiment, an associated catheter need not provide the ablation means and can simply serve to provide the expandable temporary occluding structure. Such an arrangement may be suitable in vessels that are particularly small, for instance, where the vessel diameter is less than 2 mm, or even less than 1 mm. In very narrow vessels it can be difficult to accurately deploy a catheter over the guidewire. Small vessel diameters are not uncommon in cerebrovascular indications and in oncology.

The expandable guidewire electrodes of the invention described herein provide an important advantage of being capable of collapsing at the same time as the vessel under treatment collapses. This ensures that contact between the electrode and the vessel wall is maintained during the heating phase of therapy minimising the overall time required to obtain an effective seal of the vessel, as well as ensuring greater integrity of the seal.

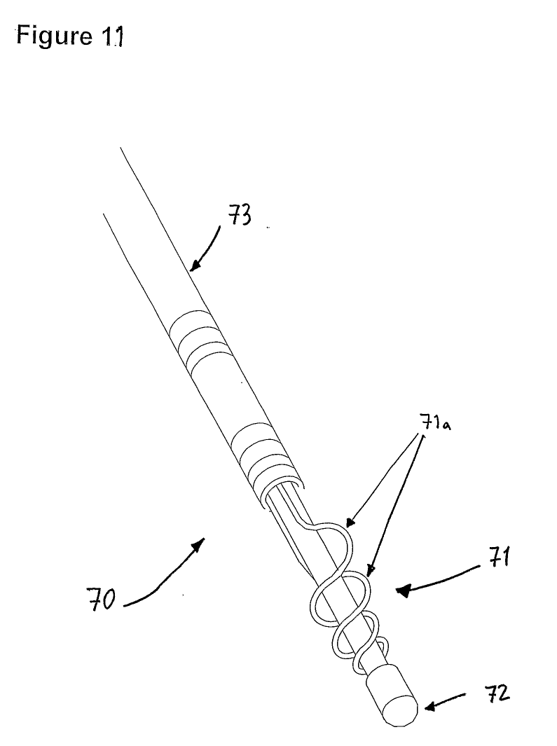

A further embodiment of the invention includes an alternative conformation for an expandable electrode located on the distal tip of the guidewire. FIG. 13(a-c) shows an expandable ‘umbrella’ electrode configuration. The guidewire 90 is provided with a tip 91 located at the distal (i.e. forward) end of a central flexible shaft 96. An annular collar 93 is slidably mounted on the shaft 96 proximally (i.e. to the rear) of the tip 91. A statically mounted hub 94 is located at a position proximally to the collar 93. Elongated flexible electrode arms 92 have an end pivotally anchored to the hub 94 and a free end 92a that extends in a distal direction. Each electrode arm 92 is either fixedly or pivotally connected to the first end of a strut 95 at an interim location 97 on the arm 92 between the hub 94 anchor point and the free end 92a. The second end of the strut 95 is pivotally anchored to the collar 93. In use, the guidewire 90 is inserted percutaneously and directed to the site where therapy is to be directed. During the insertion phase the umbrella electrode is kept in a retracted state with the electrode arms 92 held parallel to the longitudinal axis of the shaft 96, this being achieved by maximising the distance between the slidable collar 93 and the hub 94. In this configuration the free ends 92a of the arms 92 are housed within notches 98 formed in the proximally facing portion of the tip 91. When expansion of the electrode arms 92 is required, the collar 93 is drawn towards the hub 94 reducing the distance therebetween and enabling the struts 95 to bear on the arms 92 causing the free ends 92a to extend outwardly from guidewire 90 towards the surrounding vessel walls 3 (see FIG. 13(d)). In this manner the expansion of the electrode broadly mimics the opening of an umbrella.

As with the other embodiments of the invention flexible electrode arms 92 are suitably manufactured from a resilient and conductive material, for instance, stainless steel or a shape memory alloy such as nitinol. The pliability of the arms 92 is advantageous as it allows for improved contact with the vessel walls 3 and which can match the sometimes-complex surface topography over an extended area. This is particularly of advantage, for example, if the electrode is expanded for use within a varicose vein.

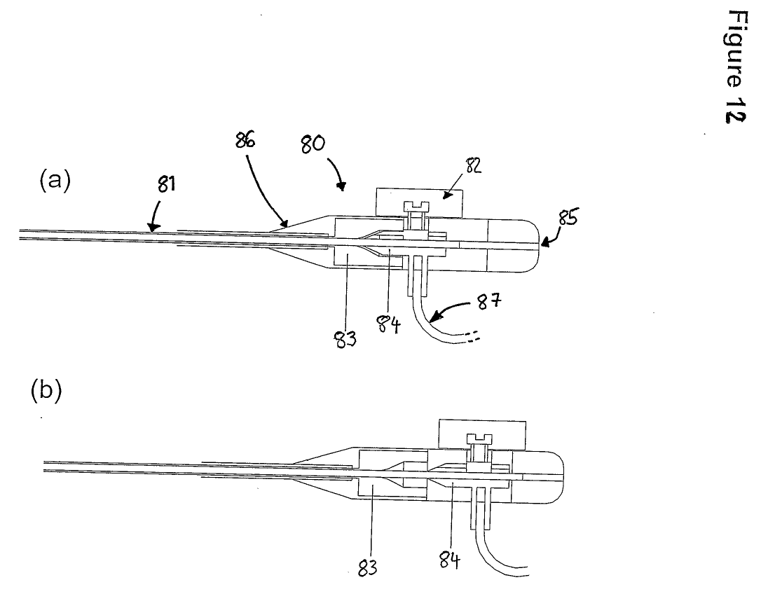

The pivotal connections between the electrode arm 92 and the hub 94, the strut 95 and the collar 93, and optionally the strut 95 and the arm 92, can suitably be in the form of an articulated joint or hinge. In a further embodiment of the invention a resilient member 94b can be located proximal to semi-fixed hub 94a (see FIG. 14(b)), this allows for the hub 94a to be displaced proximally by a certain amount in response to compression exerted on the flexible arms 92 by the contracting vessel walls during the thermal ablation step. By allowing a certain amount of free longitudinal movement of the hub 94 the contact between the arms 92 and the vessel walls 3 can be maintained, particularly if the expanded electrode is in the process of being withdrawn from the vessel whilst the ablation is occurring (as indicated by directional arrow D in FIG. 14(b)). The resilient member 94b is suitably tensioned to provide an appropriate biasing force against the hub 94a. The resilient member 94b may comprise a resilient or elastic polymeric material or a spring.

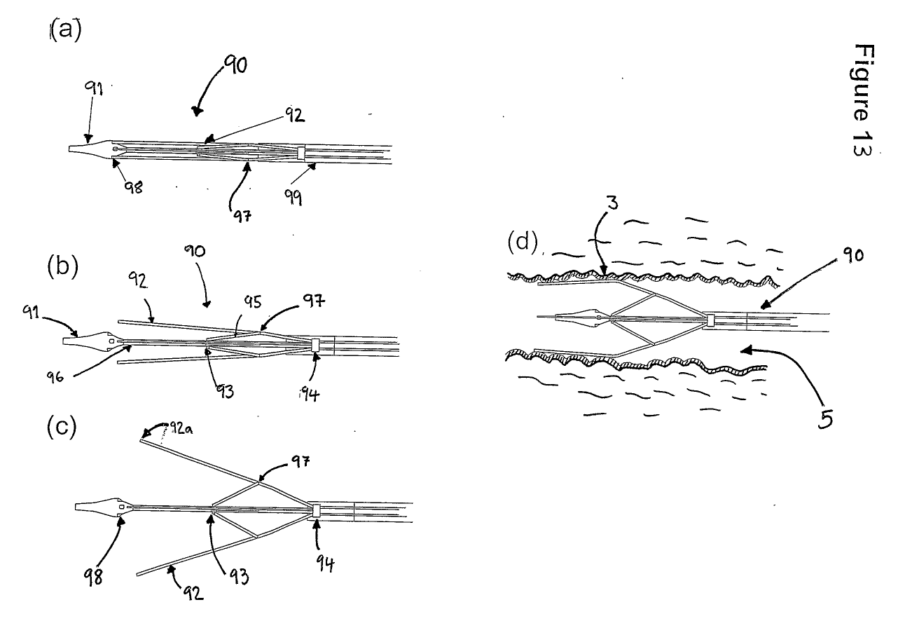

Contact between the expanded umbrella electrode and the vessel walls can be increased by inclusion of additional electrode cross wires 92b that extend across the span between adjacent expanded flexible electrode arms 92 so as to be arrayed circumferentially about the longitudinal axis of the shaft 96 (see FIG. 15 (a)). The combination of the flexible arms 92 together with the spanning cross wires 92b effectively converts the expandable electrode into an expandable web-like structure. The additional cross wires 92b are suitably manufactured from similar materials to those used to make the flexible arms 92. It should be noted that the inclusion of additional cross wires is not limited to the expandable umbrella electrode embodiment of the invention, but can also extend to the other expandable electrode configurations described above.

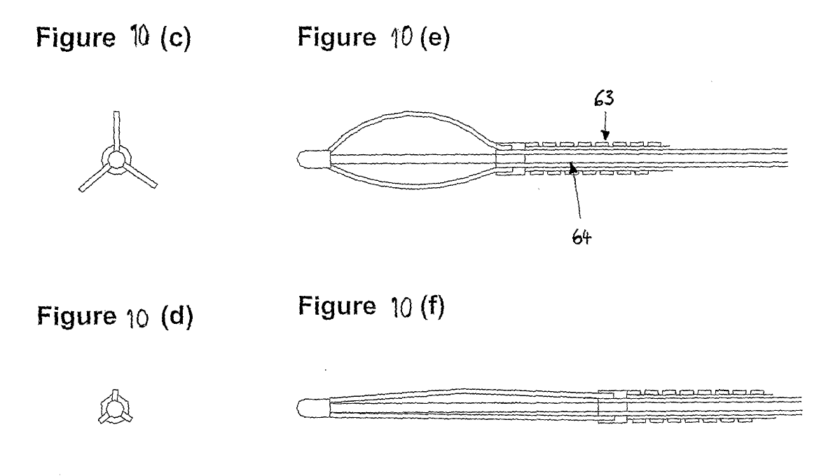

The proximal end of the device of the invention is located outside of the patient's body when in use and provides the user interface, typically in the form of a handle grip. FIGS. 12 (a) and (b) shows an embodiment of the invention in which a electrical connection of the guidewire to an RF generator is mediated via a hub 80 comprising plug and socket arrangement 83,84 that can be slidably mounted over a guidewire which passes through central channel 85 defined by the housing 86 and a barrel 81. The plug 86 is connected to an RF source via a lead 87, and can engage the socket 83 through the action of the user pushing the attached slider 82 in a distal (i.e. forward) direction. The guidewire is in electrical contact with the socket 83 (not shown) and thus when the plug 84 and socket 83 are engaged the RF source can be activated to apply RF energy at the therapy site via the guidewire or the electrode located at the distal tip of the guidewire.

In another embodiment shown in FIG. 15 one or more arms 101a can be deployed so as to protrude outwardly from the catheter 10 and penetrate through the vessel wall 3, into the tissue surrounding the vessel. When connected to an RF generator the arms can act as one or more monopolar electrodes that can ablate tissue surrounding the vessel 3. Alternatively the arms can cooperate with an expandable electrode mounted on the guidewire, with the tissue penetrating arms 101a acting as one pole of the RF electrode and guidewire providing the other pole—in a bipolar arrangement. The arms 101a can be retractably mounted in the body of the catheter 100 and deployed via apertures 102 located in the distal tip of the catheter.

In specific embodiments of the invention, the arms 101a are deployed and extend outwardly from the longitudinal axis of the catheter so as to penetrate the walls 3 of the surrounding vessel and into the tissue beyond. The arms 101a comprise a shape memory alloy that is configured such that its transition temperature is at or around the temperature at which thermal ablation is to occur (e.g. the temperature that would normally ensure endoluminal closure of the vessel to be sealed). Upon reaching the transition temperature the alignment of the arms 101a changes from one that extends outwardly to one that is substantially parallel to the longitudinal axis of the catheter. In this way the arms draw the heated tissue inwardly towards the collapsing vessel and actively contribute to the closure of the vessel. Solely by way of analogy, in this embodiment of the invention the arms 101a come together in a way that resembles the movement of the petals in a closing flower. Following the heating phase, the arms 101a can be retracted into the body of the catheter 100 via the apertures 102. Optionally a retractable sheath 110 can also be included on the catheter to shroud the distal tip portion of the catheter during the insertion and removal phases.

In an alternative embodiment of the invention one or more of the arms 101a adopts a helical conformation that spirals about the longitudinal axis of the elongate body in a distal direction through the tissue surrounding the hollow vessel. In this embodiment, similarly to the arrangement described above, it is possible to configure the arm 101a to exert additional contraction force upon the vessel during the thermal phase by manufacturing the arm 101a from a shape memory alloy. In this embodiment, the initial diameter of the helix prior to thermal ablation would be greater than the diameter assumed following transition.

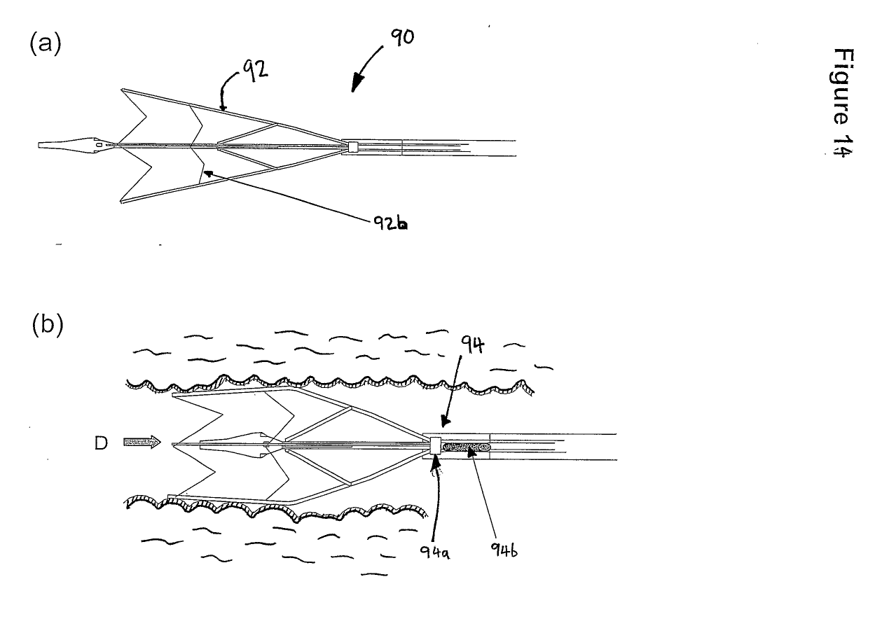

In FIG. 16 another embodiment of the invention is shown in which a bipolar RF catheter tip comprises tissue penetrating arms 122a′ and 122b extending outwardly from the respective electrodes 122a and 122b. in which two sets of arms 122a′ penetrate the vessel wall, permitting bipolar heating of the region between the two sets of arms. A retractable outer sheath 120 is further provided.

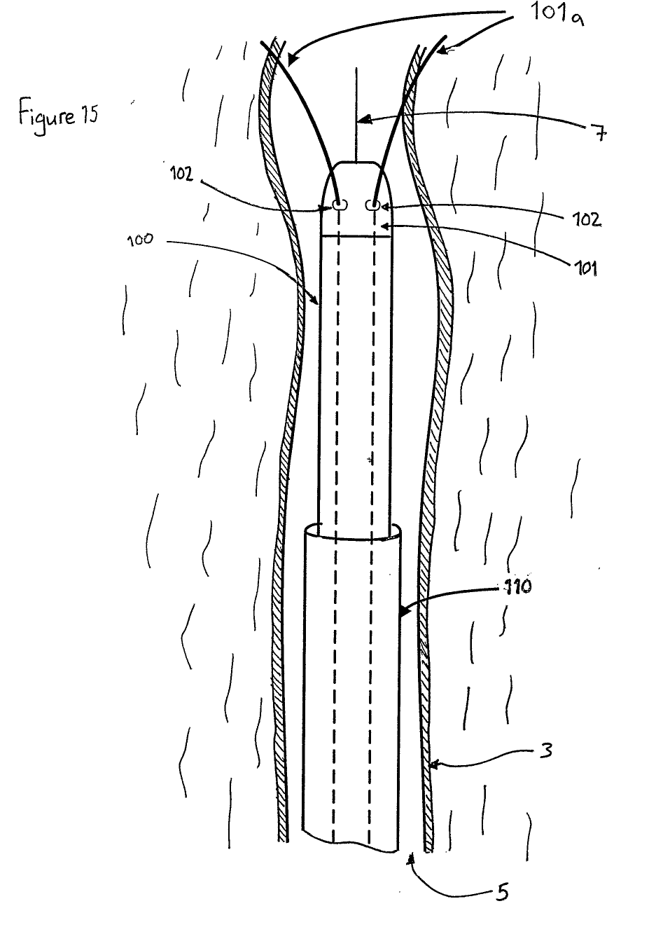

FIG. 17 shows details of penetrating arms 131a and b that can be utilised in the embodiments of the invention requiring tissue penetration. The arms 131a and b are slidably mounted in channels 133. Each channel is shaped at the distal end 134 to deflect the arms 131a and b when they are advanced in the distal direction, so the arms bend when they protrude from apertures formed in the outer body of the catheter 130, and so travel substantially perpendicular to the longitudinal axis of the catheter body towards and into the vessel wall 3.

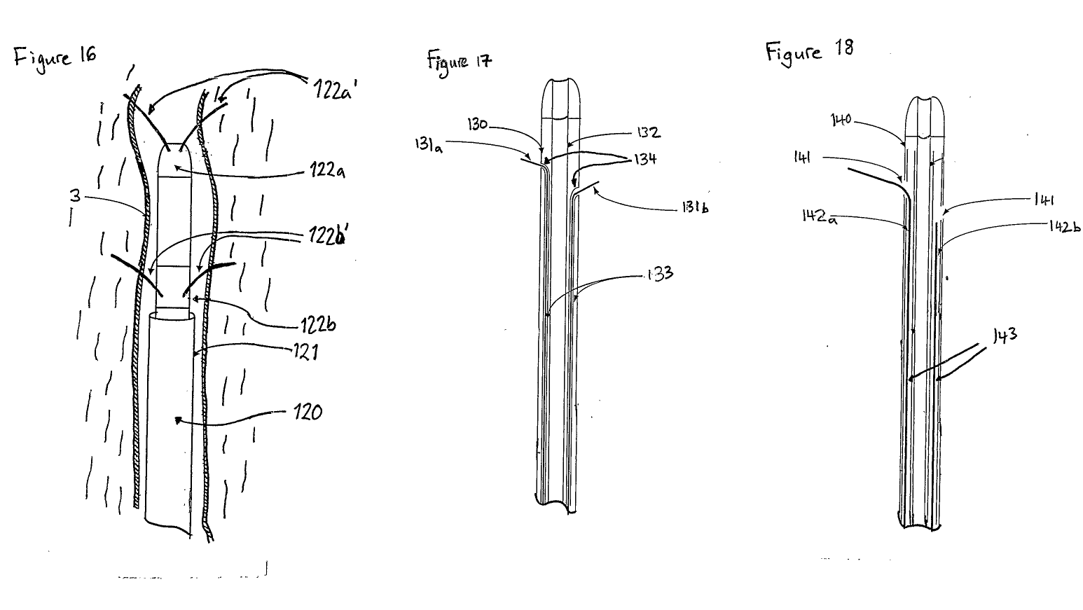

A further embodiment is shown in FIG. 18. Retractable arms 142 a and b are slidably mounted in channels 143. The channels 143 are accessible via elongated apertures or slots 141 formed in the outer sheath of the catheter. When retracted, the arm 142a and b lies in the tube substantially parallel to the elongate axis of the catheter 140. The tip of the arm is preformed to adopt a curve, so that when deployed by pushing the needle distally the arm 142a and b will exit through the slot 141, and thence into the vessel wall 3.

The tissue penetrating arms can suitably by made materials such as stainless steel, platinum, gold, silver, titanium, a metal alloy or, when required, a shape memory alloy such as nitinol.

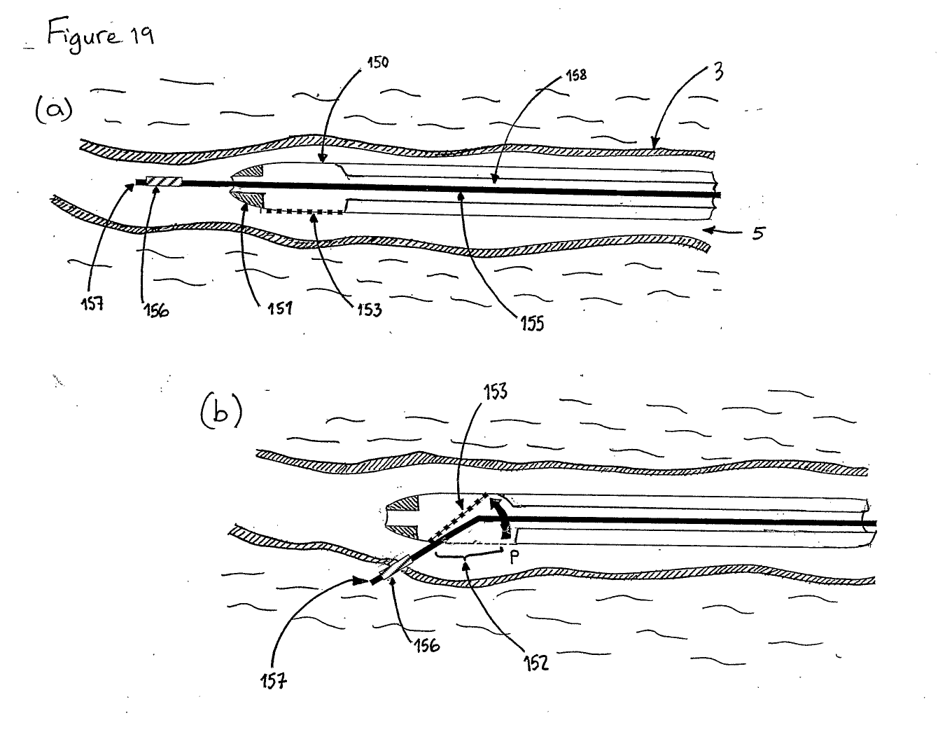

Cooperation between an RF electrode located on the catheter of the invention with another located on the guidewire is further exemplified in an alternative embodiment of the invention shown in FIG. 19. A catheter 150 is slidably mounted upon a guidewire 155. The catheter 150 comprises an RF electrode 151 located at the distal tip. An aperture 152 is positioned in the side wall of the catheter proximally to the distal electrode 151. The aperture is sealed via a pivotally mounted door 153. In use, the catheter 150 is slidably mounted onto the prepositioned guidewire 155 and located to the position in the body where therapy is required. The guidewire 155 is then withdrawn proximally until the distal tip of the guidewire 157 is withdrawn into the central lumen 158 of the catheter 150 to a point that is proximal to the aperture 152. Optionally the guidewire 155 can be withdrawn completely and substituted with a therapeutic guidewire. Retraction of the guidewire proximally causes the door 153 to open inwardly (along arrow P in FIG. 19(b)) via a user induced release mechanism (not shown) or simply by biasing the door 153 to spring open when the guidewire 155 is withdrawn. The guidewire 155 is then advanced proximally and is deflected out of the body of the catheter 150 through the aperture 152 and into the vessel wall 3. An electrode 156 on the guidewire 155 can cooperate with the electrode 151 on the catheter to allow for a bipolar RF configuration. Optionally, where the guidewire 155 is substituted for a therapeutic guidewire manufactured from a shape memory alloy or preformed material, configurations such as the helical tissue penetrating arm (described in detail above) can be adapted for use in this embodiment of the invention.

The catheters of the invention are suitably constructed in a variety of sizes typically ranging from 0.6 mm up to 2.6 mm in diameter (corresponds to French sizes 2 to 8). Guidewires of the invention are typically in the size range of 0.05 mm to about 1 mm (about 0.002 inches to about 0.05 inches). It is of considerable advantage that the design of the present catheters allows for them to be able to operate effectively in smaller vessels, since known vessel ablation catheters tend only to operate in vessels with diameters of 2.6 mm and above. Blood vessels, such as arteries, with small diameters are often found in the heart, supplying solid tumours of intermediate size and in the brain. In an embodiment of the invention, the device of the invention can be used to seal branches of the coronary artery for treatment of arrhythmia, coronary vessel anomalies or to prevent and reduce hypertrophy of the myocardium. Hence, the present invention has the advantage of providing the ability for the clinician to access and administer therapy in locations previously considered to be inaccessible to surgery. The catheters of the invention are also suitable for use in treatment of varicose veins or in stemming loss of blood from hemorrhaging tissues, including the brain, following trauma.

The invention is further exemplified by the following non-limiting examples.

### EXAMPLES

### Example 1

The device was connected to a generator via an adaptor cable, and the minimum power wattage was determined by multiple applications of the catheter in bovine liver tissue using watts between 1-40 W. This was determined to be 5 Watts.

The catheter was introduced into the liver tissue; the RF generator was set at 5 Watts and the power was applied. The timer was started in order to record the time taken for the impedance reading to increase by 10% over baseline, which was considered to be sufficient to induce tissue coagulation. When the impedance rating was reached the RF generator was placed in standby mode. The coagulated tissue was resected and zone of tissue coagulation measured. The catheter was relocated and the process was repeated a total of ten times. The results showed that there was a consistent heating region around the electrodes with no blind spots.

The results are described in the following table:

TABLE 1

| | Energy | | |
|---|---|---|---|
| | Delivered | Impedance | Ablation Time |
| Experiment | (W) | (Ohms) | (mins) |
| 1 | 5 | 414 | 0.3 |
| 2 | 5 | 569 | 0.3 |
| 3 | 5 | 598 | 0.3 |
| 4 | 5 | 589 | 0.4 |
| 5 | 5 | 564 | 0.4 |
| 6 | 5 | 614 | 0.4 |
| 7 | 5 | 522 | 0.2 |
| 8 | 5 | 555 | 0.4 |
| 9 | 5 | 517 | 0.3 |
| 10 | 5 | 552 | 0.3 |

### Example 2

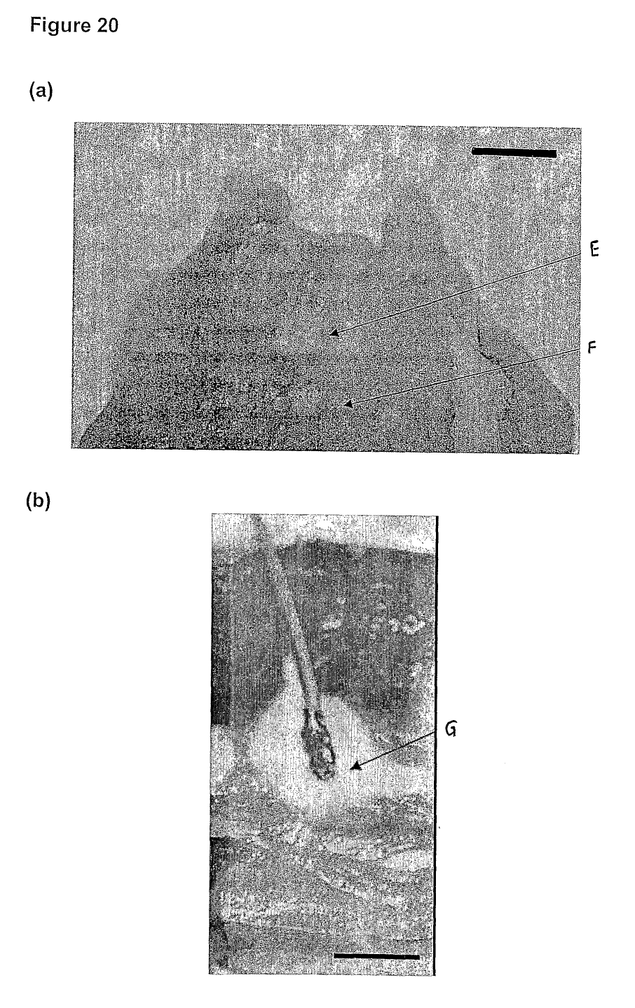

Variations between electrode distances: FIG. 20(a) shows patterns of tissue coagulation obtained with two variants of the bipolar configurations of the catheter tip in bovine liver tissue. It can be seen that at a distance of 10 mm between the proximal and distal electrodes separate and distinct ablation foci were obtained at 5 Watts of RF energy. At a distance of 7 mm between the electrodes (also at 5 Watts) the ablation foci converge to give an single elongated ablation zone. The results of a monopolar configuration are shown in FIG. 20(b), with the catheter mounted electrode being complemented with a remote grounding pad. The extensive ablation zone is indicated by arrow G.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1. A device suitable for percutaneous insertion into a hollow vessel for purpose of causing endoluminal closure of the vessel at a specified therapeutic site in the body of a patient, comprising:
an elongate body having a distal end and a proximal end, the distal end comprising a distal tip portion, and a central lumen extending along at least a portion of the length of the elongate body, wherein the central lumen is configured to enable slidable mounting of the device upon a prelocated guidewire;
the distal tip portion comprising at least one heating module capable of heating the walls of the hollow vessel to a temperature that causes endoluminal closure of the hollow vessel; and
wherein the distal tip portion further comprises at least one extendable element that can be deployed outwardly from the elongate body so as to contact and/or penetrate the walls of the hollow vessel.

2. The device of claim 1, wherein the central lumen extends along the entire length of the device, thereby facilitating an over-the-wire mounting on the pre-located guidewire.

3. The device of claim 1, wherein the central lumen extends along a portion of the device, thereby facilitating a monorail mounting on the pre-located guidewire.

4. The device of claim 1, wherein the at least one heating module comprises a heating element selected from the group consisting of: a bipolar radiofrequency (RF) electrode arrangement; a monopolar RF electrode arrangement; a microwave energy source; and ultrasound energy source; and a laser energy source.

5. The device of claim 4, wherein the at least one heating module comprises a bipolar RF electrode arrangement, comprising a first electrode located at the distal tip of the elongate body and a second electrode located at a position proximally to the first electrode.

6. The device of claim 5, wherein the first and second electrodes are spaced apart by a distance of not more than about 15 mm.

7. The device of claim 5, wherein the first and second electrodes are spaced apart by a distance of not more than about 12 mm.

8. The device of claim 5, wherein the first and second electrodes are spaced apart by a distance of not more than about 10 mm.

9. The device of claim 5, wherein the first and second electrodes are spaced apart by a distance of not more than about 7 mm.

10.-77. (canceled)

78. The device of claim 1, wherein the extendable element is associated with the at least one heating module.

79. The device of claim 1, wherein the extendable element is selected from the group consisting of: a wire; an arm; a panel; and a needle.

80. The device of claim 78, wherein the at least one heating module comprises at least one RF electrode, wherein the at least one RF electrode further comprises at least one extendable element that can be extended outwardly from the elongate body so as to contact the walls of the hollow vessel.

81. The device of claim 78, wherein the at least one heating module comprises at least one RF electrode, wherein the at least one RF electrode further comprises at least one extendable element that can be extended outwardly from the elongate body so as to penetrate the walls of the hollow vessel.

82. The device of claim 1, wherein the wherein the elongate body of the device comprises an outer sheath, and wherein elongate body further comprises at least one channel extending along its length, which channel can accommodate the at least one extendable element in a retracted configuration, such that the extendable element can be extended outwardly from the elongate body via an aperture in the outer sheath when the device is correctly located at the specified site in the body of the patient.

83. The device of claim 1, wherein the distal tip portion is can be shrouded within a retractable outer sheath, thereby constraining the at least one extendable element, such that when the device is correctly located at the specified site in the body of the patient, the outer sheath can be retracted proximally thereby allowing the at least one extendable element to extend outwardly.

84. The device of claim 1, wherein the extendable element comprises an electrically conductive material that is pre-stressed so that in its unconstrained state it extends outwardly from the longitudinal axis of the elongate body of the device.

85. The device of claim 1, wherein the extendable element comprises a material selected from one of gold; platinum; silver; a metal alloy; a shape memory alloy; stainless steel; and titanium.

86. The device of claim 1, wherein the extendable element comprises the shape memory alloy nitinol.

87. The device of claim 1, wherein the extendable element comprises at least one arm that can extend outwardly from the longitudinal axis of the elongate body so as to penetrate the walls of the hollow vessel, the at least one arm comprising a shape memory alloy that is configured such that its transition temperature is at or around the temperature that causes endoluminal closure, and upon reaching the transition temperature the alignment of the at least one arm changes from one that extends outwardly from the longitudinal axis of the elongate body to one that is substantially parallel to the longitudinal axis of the elongate body.

88. The device of claim 1, wherein the extendable element comprises a plurality of arms.

89. The device of claim 1, wherein the extendable element comprises at least one arm that can extend outwardly from the longitudinal axis of the elongate body so as to penetrate the walls of the hollow vessel, wherein upon extension the at least one arm adopts a helical conformation that spirals about the longitudinal axis of the elongate body in a distal direction through the tissue surrounding the hollow vessel.

90. The device of claim 5, wherein the first and/or second electrodes are made from a material selected from one of the group consisting of stainless steel; silver; gold; platinum; titanium; a metal alloy; and a shape memory alloy.

91. The device of claim 4, wherein the at least one heating module comprises a monopolar RF electrode arrangement, comprising a first electrode located in the distal tip portion of the elongate body that cooperates with a second electrode, located externally to the patient's body, in order to complete the RF circuit.

92. The device of claim 4, wherein the at least one heating module comprises a monopolar RF electrode arrangement, comprising a first electrode located in the distal tip portion of the elongate body that cooperates with a second electrode located at a position on the guidewire that is adjacent to the distal tip portion of the elongate body when at the specified therapeutic site in the body of the patient, in order to complete an RF circuit.

93. The device of claim 92, wherein the guidewire comprises a distal tip portion and the second electrode is located on the distal tip.

94. The device of claim 92, wherein the guidewire comprises a distal tip portion and the second electrode is located at a position adjacent and immediately proximal to the distal tip.

95. The device of claim 92, wherein the second electrode comprises an expandable structure that when deployed is capable of contacting the walls of the hollow vessel.

96. The device of claim 92, wherein the second electrode comprises an expandable structure that when deployed is capable of contacting the walls of the hollow vessel, and wherein the expandable structure is selected from one of the group consisting of: an umbrella structure; a single helical coil; a double helical coil; and an expandable basket.

97. The device of claim 1, further comprising an expandable occlusion structure located on the elongate body at a position that is proximal to the distal tip portion and which can be expanded outwardly from the elongate body so as to cause temporary occlusion of the hollow vessel.

98. The device of claim 1, wherein the distal tip portion further comprises a temperature sensor.

99. The device of claim 1, wherein the hollow vessel is a blood vessel.

100. A device suitable for percutaneous insertion into a hollow vessel for purpose of causing endoluminal closure of said vessel at a specified therapeutic site in the body of a patient comprising:
a guidewire having a distal end and a proximal end, the distal end comprising a first distal tip portion, and wherein the distal tip portion comprises a first RF electrode located at a position adjacent and immediately proximal to the distal end; and
an elongate body having a distal end and a proximal end, the distal end comprising a second distal tip portion, and a central lumen extending along a least a portion of the length of the elongate body, wherein the central lumen is configured to enable slidable mounting of the elongate body upon the guidewire, and the second distal tip portion comprising a second RF electrode;
wherein in use the guidewire and elongate body are juxtaposed such that upon application of RF energy the first and second RF electrodes are capable of cooperating to cause heating the walls of the hollow vessel to a temperature that causes endoluminal closure of the hollow vessel.

101. The device of claim 100, wherein the second electrode comprises an expandable structure that when deployed is capable of contacting the walls of the hollow vessel.

102. The device of claim 101, wherein the expandable structure is selected from one of the group consisting of: an umbrella structure; a single helical coil; a double helical coil; an expandable basket.

103. The device of claim 100, wherein the distal tip portion further comprises at least one extendable element that can be deployed outwardly from the longitudinal axis of the elongate body so as to contact and/or penetrate the walls of the hollow vessel.

104. The device of claim 103, wherein the extendable element comprises at least one arm that can extend outwardly from the elongate body so as to contact the walls of the hollow vessel.

105. The device of claim 103, wherein the extendable element comprises at least one arm that can extend outwardly from the elongate body so as to penetrate the walls of the hollow vessel.

106. The device of any of claim 103, wherein the distal tip portion further comprises an outer sheath and wherein the elongate body comprises at least one channel extending along its length, which channel can accommodate the extendable element in a retracted configuration, such that the extendable element can be extended outwardly from the elongate body via an aperture in the outer sheath when the device is correctly located at the specified site in the body of the patient.

107. The device of claim 103, wherein the distal tip portion further comprises a retractable outer sheath that serves to shroud the distal tip portion, thereby constraining the extendable element, such that when the device is correctly located at the specified site in the body of the patient, the outer sheath can be retracted proximally thereby allowing the extendable element to extend outwardly.

108. The device of claim 103, wherein the extendable element comprises an electrically conductive material that is pre-stressed so that in its unconstrained state it extends outwardly from the longitudinal axis of the elongate body of the device.

109. The device of claim 103, wherein the extendable element comprises a material selected from one of the group consisting of: gold; platinum; silver; a metal alloy; a shape memory alloy; stainless steel; and titanium.

110. The device of claim 103, wherein the extendable element comprises the shape memory alloy nitinol.

111. The device of claim 103, wherein the extendable element comprises at least one arm that can extend outwardly from the longitudinal axis of the elongate body so as to penetrate the walls of the hollow vessel, the at least one arm comprising a shape memory alloy that is configured such that its transition temperature is at or around the temperature that causes endoluminal closure, and upon reaching the transition temperature the alignment of the at least one arm changes from one that extends outwardly from the longitudinal axis of the elongate body to one that is substantially parallel to the longitudinal axis of the elongate body.

112. The device of claim 103, wherein the extendable element comprises at least one arm that can extend outwardly from the longitudinal axis of the elongate body so as to penetrate the walls of the hollow vessel, wherein upon extension the at least one arm adopts a helical conformation that spirals about the longitudinal axis of the elongate body in a distal direction through the tissue surrounding the hollow vessel.

113. The device of claim 100, wherein the elongate body comprises an aperture positioned in a side wall of the elongate body proximally to the distal tip region, the aperture being sealed via an inwardly pivoting door, such that in use the door is displaced outwardly so as to seal the aperture when the device is loaded onto the guidewire, wherein upon withdrawal of the guidewire in a proximal direction where the distal tip of the guidewire is located in the central lumen at a position that is proximal to the aperture, the door can be opened inwardly such that when the distal tip of the guidewire is subsequently advanced distally the distal tip of the guidewire is deflected outwardly from the elongate body of the device by the door and through the aperture into the wall of the surrounding hollow vessel.

114. A method for endoluminal closure a blood vessel at a predetermined site within the body of a patient, the predetermined site being within or adjacent to the site of a lesion in tissue that is supplied by the blood vessel, the method comprising:
(a) introducing into the blood vessel a guidewire at a site remote from the predetermined site within the body of the patient, the guidewire having a distal tip, and directing the distal tip of the guidewire to a location substantially within the vicinity of the predetermined site;
(b) introducing onto the guidewire via a slidable mounting, a catheter, wherein the catheter comprises a distal tip region comprising at least one heating module located thereon;
(c) directing the distal tip region of the catheter to the predetermined site within the body of the patient by tracking the catheter along the guidewire;
(d) applying energy to the walls of the blood vessel via the heating module such that the tissue is heated to a point that causes endoluminal closure of the blood vessel;
(e) monitoring the energy application in step (d);
(d) ceasing application of energy when endoluminal closure has been completed; and
(e) withdrawing the catheter and guidewire from the closed blood vessel.

115. The method of claim 114, wherein the heating module comprises a heating element selected from the group consisting of: a bipolar radiofrequency (RF) electrode arrangement; a monopolar RF electrode arrangement; a microwave energy source; and ultrasound energy source; and a laser energy source.

116. The method of claim 114, wherein the at least one heating module comprises a bipolar RF electrode arrangement, comprising a first electrode located at the distal tip of the elongate body and a second electrode located at a position proximally to the first electrode.

117. The method of claim 114, wherein the lesion is selected from the group consisting of: a solid tumor; traumatized tissue; hemorrhaging tissue; infected tissue; and anatomically aberrant tissue.

118. The method of claim 114, wherein step (e) comprises monitoring a change in electrical impedance of the tissue in the walls of the blood vessel during the energy application stage.

119. The method of claim 114, wherein step (e) comprises monitoring a change in temperature of the tissue in the walls of the blood vessel during the energy application stage.
